SEQUENCE LISTING (9 sequences)

SEQ ID NO: 1 - DNA, 42 bp, Artificial Sequence
  ADAmF primer
gttggygtataaagttgtttttatttattgagtatttagtag

SEQ ID NO: 2 - DNA, 37 bp, Artificial Sequence
  ADAmR primer
ctcaatttcrctatctatcaaataaacatcctaacac

SEQ ID NO: 3 - DNA, 35 bp, Artificial Sequence
  VHLmF primer
gatagatgtaaagataggaataagttagggttatg

SEQ ID NO: 4 - DNA, 32 bp, Artificial Sequence
  VHLmR primer
ctccraaaaatactccttaactaaaaccacac

SEQ ID NO: 5 - DNA, 33 bp, Artificial Sequence
  CD3ZmF primer
tttggggaggtagttgtagaataaaattagtag

SEQ ID NO: 6 - DNA, 25 bp, Artificial Sequence
  CD3ZmR primer
cacacccactcccctacacatacac

SEQ ID NO: 7 - DNA, 34 bp, Artificial Sequence
  ADA-SBE1 primer
ctacnccnaataaacacctaatactatacccnac

SEQ ID NO: 8 - DNA, 35 bp, Artificial Sequence
  VHL-E1 primer
gatgtaaagataggaataagttagggttatgttgg

SEQ ID NO: 9 - DNA, 26 bp, Artificial Sequence
  CD3Z-E1 primer
gggaaaggataagatgaagtggaagg

(19) United States
(12) Patent Application Publication
(10) Pub. No.: US 2012/0178633 A1
(43) Pub. Date: Jul. 12, 2012

(54) DIAGNOSTIC COMPOSITION FOR AUTOIMMUNE DISEASES COMPRISING AGENT MEASURING CD3Z GENE METHYLATION LEVEL AND A METHOD FOR DIAGNOSING AUTOIMMUNE DISEASES USING THE SAME

(75) Inventors: Kyeong Man HONG, Gyeonggi-do (KR); Yeong Wook SONG, Seoul (KR)

(73) Assignees: SNU R&DB FOUNDATION, Seoul (KR); NATIONAL CANCER CENTER, Gyeonggi-do (KR)

(21) Appl. No.: 13/339,462

(22) Filed: Dec. 29, 2011

(30) Foreign Application Priority Data
Dec. 29, 2010 (KR) 10-2010-0138285

(51) Int. Cl. C12Q 1/68; C01D 5/00; C40B 20/00; C12N 9/16; C07H 21/04

(52) U.S. Cl. 506/2; 435/196; 536/24.33; 435/6.11; 423/519

(57) ABSTRACT

The present invention relates to a diagnostic composition for autoimmune diseases, comprising agent measuring a methylation level of CD3Z gene, a diagnostic method and a kit using the same. More particularly, the present invention relates to a composition for diagnosing autoimmune diseases according to the methylation level of CD3Z gene, or additionally ADA or VHL gene, and a method for diagnosing autoimmune diseases by measuring the methylation level. The methylation of any one or more of the ADA, VHL, and CD3Z genes of the present invention is specific to autoimmune diseases, and thus the composition comprising an agent measuring a methylation level of the present invention can be used for the diagnosis of autoimmune diseases.

### BACKGROUND OF THE INVENTION

1. Field of the Invention

The present invention relates to a diagnostic composition for diagnosing autoimmune diseases, comprising an agent measuring a methylation level of CD3Z gene, a diagnostic method and a kit using the same. More particularly, the present invention relates to a composition for diagnosing autoimmune diseases according to the methylation level of CD3Z gene, or one or more genes selected from ADA, VHL and CD3Z genes, and a method for diagnosing autoimmune diseases by measuring the methylation level.

2. Description of the Related Art

Autoimmune diseases are those that occur as a result of the body's immune system attacking normal, healthy tissues, organs or other body parts. Many autoimmune diseases are attributed to an overactive immune response of the body, and cause self destruction. Well-known examples thereof include rheumatoid arthritis (RA), systemic sclerosis (SSC), systemic lupus erythematosus (SLE), sclerosis, polymyositis, dermatomyositis, Henoch-Shoenlein Purpura, Sjogren's syndrome, etc. Other diseases such as primary biliary cirrhosis (PBC), chronic active hepatitis, and Hashimoto's thyroiditis are also related to autoimmune diseases.

Rheumatoid arthritis (RA) is an autoimmune diseases that causes a chronic systemic inflammation affecting the joints, and is more prevalent in women than men. Although the cause is not clearly understood yet, both genetic and environmental factors are believed to contribute to the disease process. Many studies have been made attempting to elucidate the cause of rheumatoid arthritis. Recently, it has been reported that genetic factors play an important role in the diagnosis of rheumatoid arthritis and imbalance of epigenetic control over immune response may contribute to the pathogenesis of autoimmune diseases including rheumatoid arthritis. Until now, the diagnosis of rheumatoid arthritis has been usually based on criteria established by the American College of Rhematology. The percentage positive of a rheumatoid factor as an objective index is about 33% within three months and about 88% in twelve months and longer, and a definite diagnosis of rheumatoid arthritis has not been achieved.

Systemic sclerosis (SSC) is commonly called scleroderma, and is characterized by functional and structural abnormalities of small blood vessels, fibrosis of the skin and internal organs, immune system activation, and auto immunity. The diagnosis of systemic sclerosis is based on criteria established by the American College of Rhematology which looks for the following: 1) proximal scleroderma: symmetric thickening, tightening, and induration of the skin of the fingers and the skin proximal to the metacarpophalangeal or metatarsophalangeal joints, and the changes may affect the face, neck, thorax, and abdomen, 2) sclerodactyly, 3) digital pitting scars or tissue loss of the volar pads of the fingertips, and 4) bibasilar pulmonary fibrosis.

Systemic lupus erythematosus (SLE) is a chronic inflammatory connective tissue disorder that can involve joints, kidneys, mucous membranes, and blood vessel walls and is potentially lethal. This disease is often abbreviated as lupus. Symptoms and signs in the joints, nervous system, blood, skin, kidneys, gastrointestinal tract, and other tissues and organs can develop. About 70 to 90% of people who have lupus are young women in their late teens to 30s, but children (mostly girls) and older men and women can also be affected. Lupus occurs in all parts of the world, but may be more common among blacks and Asians.

The cause of lupus is usually not known. Occasionally, the use of certain drugs such as hydralazine, procainamide, and isoniazid can cause lupus. Drug-induced lupus usually disappears after the drug is discontinued. Symptoms vary greatly from person to person. Symptoms may begin suddenly with a fever, may develop gradually over months or years with flare-ups, neuropsychiatric manifestations, or any of the symptoms associated with lupus.

Lupus tends to be chronic and relapsing, often with symptoms-free periods that can last for years. Flare-ups can be triggered by sun exposure, infection, surgery, or pregnancy. Flare-ups occur less often after menopause. In a six-year prospective cohort study, disease flares occurred at a rate of 0.2 per year per patient. Because the course of lupus is unpredictable, the prognosis varies widely. A cohort study found that within seven years of diagnosis, 61% of patients developed clinically detectable organ damage, with neuropsychiatric (20.5%), musculoskeletal (18.5%), and renal (15.5%) organ systems most commonly affected. However, if the initial inflammation is controlled, the long-term prognosis is good. The major causes of death in patients with SLE are cardiovascular, renal, lung, and CNS infections and active diseases. Early detection and treatment of kidney damage reduces the incidence of severe kidney disease. Accurate diagnosis of SLE is important because treatment can reduce morbidity and mortality, particularly from lupus nephritis. However, the disease has no single diagnostic marker; instead, it is identified through a combination of clinical and laboratory criteria. The laboratory criteria set developed by the ACR (American College of Rheumatology) is most widely used. Elevation of the antinuclear antibody (hereinafter, referred to as ANA), titers to 1:40 or higher, is the most sensitive of the ACR diagnostic criteria. More than 99 percent of patients with SLE have an elevated ANA titer at some point, although a significant proportion of patients may have a negative ANA titer early in the disease. However, the ANA test is not specific for SLE. A study involving international laboratories found that ANA tests in the general population were positive in 32% of persons at a 1:40 dilution and in 5% of persons at a 1:160 dilution. An ANA titer of 1:40 or higher has a positive predictive value of only 10% because of the common occurrence of high ANA titers in children.

In the absence of SLE, the most common reason for a positive ANA test is the presence of another connective tissue disease. Diseases that are often associated with a positive ANA test include Sjogren's syndrome, scleroderma, rheumatoid arthritis, and juvenile rheumatoid arthritis. An ANA test also can be positive in patients with fibromyalgia. In patients with diseases other than SLE, ANA titers usually are lower, and the immunofluorescent pattern is different. Rates of positive ANA tests are affected by the prevalence of SLE in the population.

Testing for antibody to double-stranded DNA antigen (anti-dsDNA) and antibody to Sm nuclear antigen (anti-Sm) may be helpful in patients who have a positive ANA test but do not meet the full criteria for the diagnosis of SLE. Anti-dsDNA and anti-Sm, particularly in high titers, have high specificity for SLE, although their sensitivity is low. Therefore, a positive result helps to establish the diagnosis of the disease, but a negative result does not rule it out.

In recent years, increasing evidence has demonstrated the role of epigenetic alterations in the etiology of many diseases. For instance, unscheduled hypermethylation of CpG islands of tumor suppressor genes and the resulting transcriptional silencing are associated with malignant transformation in cancer. Other diseases with well-recognized epigenetic defects include ICF (Immunodeficiency, Centromeric region instability, and Facial anomalies) syndrome, Prader-Willi and Beckwith-Wiedemann syndromes, and Rett syndrome. In fact, the epigenetic framework could explain several characteristics of many diseases, including their age dependence and quantitative nature, and the mechanism by which the environment modulates genetic predisposition to disease. Moreover, recent findings have indicated that epigenetic alterations accumulate gradually over an individual's lifetime. In fact, the comparison of epigenetic modifications in genetically identical monozygotic twins has revealed that environmental factors, including diet and lifestyle, contribute significantly to the phenotype by changing the epigenetic profile. Individual epigenetic peculiarities that modulate susceptibility could therefore explain the apparent complexity in the patterns of inheritance.

Besides the well-recognized genetic susceptibility to SLE, epigenetic factors have recently received much attention, since it was reported that T cells from patients with active lupus were shown to exhibit globally hypomethylated DNA. The first evidence of the role of aberrant changes in the DNA methylation patterns in the development of SLE was that T cells from patients with active lupus were shown to exhibit globally hypomethylated DNA. More recent studies have demonstrated an association between DNA hypomethylation in SLE and a decrease in, the enzymatic activity of DNMTs, implying a possible mechanism to explain DNA hypomethylation. Additional evidence of the role of methylation changes in the development of SLE comes from studies with DNA demethylating drugs. One of the most common demethylating drugs used to induce SLE in mice is 5-azacytidine, a cytosine analog that contains a nitrogen atom at the 5′ position of the pyrimidine ring and is incorporated into newly synthesized DNA. Treatment with 5-azacytidine causes genome-wide hypomethylation, resulting in the altered expression of many genes. Other demethylating drugs used to induce SLE are procainamide, a competitive DNMT inhibitor, and hydralazine, whose demethylating activity has been explained as an indirect result of the inhibition of the ERK pathway signaling, decreasing DNMT1 and DNMT3a levels during mitosis. In all cases, exposing T cells to demethylating drugs results in the demethylation-dependent induction of lupus-like disease.

The identification of genes that are deregulated through DNA methylation changes in SLE contributes to the understanding of the pathway of the disease. Recently, specific promoter demethylation of several genes in SLE has been shown to contribute to aberrant overexpression of various genes. These aberrant changes occur in genes like perforin (Kaplan M J, et al., Demethylation of promoter regulatory elements contributes to perforin overexpression in CD4+lupus T cells. J Immunol. 172(6):3652-61.(2004)), whose demethylation could contribute to monocyte killing. CD70 is also overexpressed in CD4+ lupus T cells by demethylation (Lu, Q. et al., Demethylation of ITGAL (CD11a) regulatory sequences in systemic lupus erythematosus. Arthritis Rheum. 46: 1282-1291. (2002). In this case, CD70 overexpression contributes to excessive B cell stimulation in lupus. Another example is the demethylation of ITGAL regulatory sequences, which may also contribute to the development of lupus. Recent DNA methylation array study of identical twins discordant for SLE also revealed that the promoter demethylation of several genes are associated with incidence and progression of SLE (Javierre B M. et al., Changes in the pattern of DNA methylation associate with twin discordance in systemic lupus erythematosus. Genome Res. February; 20(2):170-9. (2010) Epub 2009 Dec. 22), suggesting epigenetic changes may be critical in the clinical manifestation of autoimmune disease.

There are many reports on a relationship between hypomethylation and SLE. On the contrary, there is little report on hypermethylation in autoimmune diseases including SLE, and there have been no case-control studies showing the role of methylation changes in autoimmune diseases including SLE.

Therefore, the present inventors have made many efforts to develop DNA markers specific to autoimmune diseases, and they found the methylation patterns of CD3Z, ADL, and VHL genes specific to autoimmune diseases, and a possibility of diagnosing autoimmune diseases by measuring the methylation level, thereby completing the present invention.

### SUMMARY OF THE INVENTION

An object of the present invention is to provide a diagnostic composition for autoimmune diseases, comprising an agent measuring a methylation level of CD3Z gene (CD3 zeta, NCBI GenBank Accession No. NM_{—}198053.2).

Another object of the present invention is to provide a method for diagnosing autoimmune diseases, comprising the steps of measuring a methylation level of CD3Z gene (CD3 zeta, NCBI GenBank Accession No. NM_{—}198053.2) in a biological sample of a patient suspected of having autoimmune diseases; and comparing the methylation level to that of the corresponding gene in a normal control group.

Still another object of the present invention is to provide a diagnostic kit for autoimmune diseases, comprising the composition.

### BRIEF DESCRIPTION OF THE DRAWINGS

FIG. 1 is a schematic diagram showing a principle of MSBE (multiple single-base extension).

FIG. 2 is the results of bisulfite sequencing (sodium bisulfite sequencing) of promoter methylation of CD3Z, ADA and VHL, in which gDNAs of patients were modified by treatment with sodium bisulfite using an EZ DNA Methylation kit (Zymo Research), each sequence was amplified using the modified gDNA as a sample and primers specific thereto, followed by cloning and determination of base sequence. In the result of base sequence determination, C was determined as methylation, and T was determined as unmethylation. The methylated CpG nucleotides are indicated by the filled circles, and unmethylated CpGs, by the open circles. Methylation sequencing from bisulfite-modified DNA was performed on gDNAs of 6 persons showing different methylation levels in the result of MSBE (multiple single-base extension). Methylation single base extension results for the three genes are shown as CD3Z, ADA, and VHL, respectively. This result was obtained by modifying each gDNA by treatment with bisulfite, amplifying the sequence corresponding to each gene using primers for CD3Z, ADA and VHL genes described in Table 1, and cloning and determining the base sequence. In the result, the methylated CpG sites are indicated by the filled circles, and unmethylated CpG sites, by the open circles. ATG is the start site of protein synthesis. For quantitative comparison between MSBE and bisulfite sequencing, the methylation levels {M/(M+U)} obtained from the MSBE results of six samples were marked in the side of each sample number, and the results are compared to the percentages of the filled circles. A significant correlation was observed in all three genes of CD3Z, ADA, and VHL (Pearson correlation coefficient R=0.922(ADA), 0.980(CD3Z) or, 0.970(VHL) p=0.009(ADA), 0.001(CD3Z), or 0.001(VHL)).

FIG. 3 is the MSBE results of quantifying CpG island methylation of CD3Z, ADA and VHL genes, showing that SLE patients have higher promoter methylation, in which M/(M+U) was calculated from signal intensities of methylated (M) and unmethylated (U) peaks, and defined as methylation level, used for further analysis.

FIG. 4 shows quantitative changes in promoter CpG island methylation levels of A. CD3Z, B. ADA, and C. VHL, in which SLE patients showed significantly high values (p<0.01 by Wilcoxon rank sum test) in the promoter CpG island methylation of three different genes, compared to the healthy control group (NL), the Y axis represents a signal ratio of a methylated peak (M) to an unmethylated peak (U), and SLE patients showed significant changes (p<0.01 by Wilcoxon rank sum test) in the promoter methylation of CD3Z, ADA and VHL genes, compared to the healthy control group.

FIG. 5 shows the methylation levels of CD3Z and VHL in 4 pairs of twins discordant for SLE, in which increased methylation levels were observed in SLE patients, compared to non-patient siblings.

FIG. 6 shows the promoter methylation of CD3Z, ADA, and VHL genes, when SLE blood was separated into the whole blood (WB), the mononucleated cell (MNC) and the granulocyte (Gran). The results of relative promoter methylation in the mononucleated cell or the granulocyte of SLE patients are shown as A.CD3Z, B. ADA, and C. VHL. Most of CD3Z gene was methylated in the granulocyte. Other methylation changes in the whole blood were similar to those in the mononucleated cell or the granulocyte.

FIG. 7 shows the relationship between promoter methylation of CD3Z, ADA, and VHL genes and gene expression in cell lines. The promoter CpG island methylation of CD3Z gene in HCC-95 and HCC-1833 cell lines, that of ADA gene in HCC-95 and HCC-1588 cell lines, and that of VHL gene in TK10 and 786-0 cell lines were observed, and in each case, there was no expression of each gene. However, the expression of each gene was restored after treatment of the cell lines with a demethylating agent 5-aza deoxy cytidine. In this regard, GAPDH was used as a control.

FIG. 8 shows quantitative changes of TCRζ-chain positive CD3 cell in SLE patients. The TCRζ expression index was determined by flow cytometry, and the experiment was performed, based on the MFI index and the TCRζ^{bright}/^{dim} ratio. The MFI index is calculated by dividing the number of MFI ^{TCRζ}^{postive} cells by the number of MFI TCRζ^{negative} cells in A. The TCRζ^{bright}/TCRζ^{dim} ratio is calculated by dividing the number of TCRζ^{bright} cells by the number of TCRζ^{dim} cells. A shows flow cytometry data of normal control group (NR) or SLE patients. B shows the TCRζ^{bright}/TCRζ^{dim} ratio of normal control group (NR) or SLE patients. C shows the MFI index of normal control group (NR) or SLE patients. Down-regulation of CD3Z protein product in SLE was observed, and for examination of TCRζ-chain, the expression levels of TCRζ-chain were examined in healthy controls and SLE patients. Compared to the normal healthy controls, there was no difference in the MFI index between SLE patients and controls, but a significant decrease in the TCRζ^{bright}/^{dim} ratio was observed in SLE patients (p<0.001, by Mann-Whitney U test).

FIG. 9 shows that the promoter methylation of CD3Z gene is inversely related to the TCRζ-chain expression on T cell surface, and the TCRζ^{bright}/^{dim} ratio of 21 healthy controls determined by flow cytometry was in inverse proportion to the CD3Z methylation level, namely, M/(M+U) (p=0.0140 by Spearman correlation test).

### DETAILED DESCRIPTION OF THE PREFERRED EMBODIMENTS

In one embodiment to achieve the above objects, the present invention relates to a diagnostic composition for autoimmune diseases, comprising an agent measuring a methylation level of CD3Z gene (CD3 zeta, NCBI GenBank Accession No. NM_{—}198053.2).

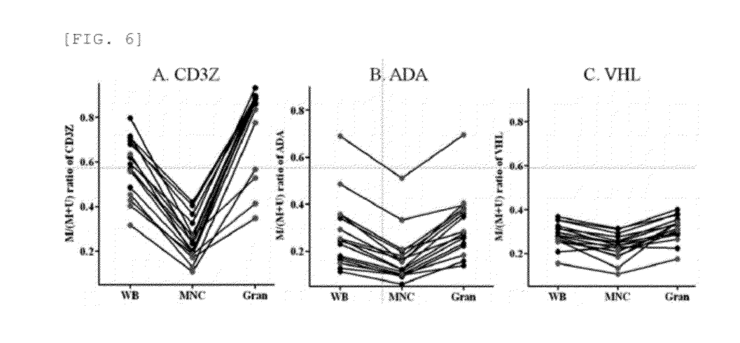

Autoimmune diseases can be diagnosed by measuring the methylation level of the CD3Z gene alone. However, preferably the methylation level of ADA (Adenosine deaminase, NCBI GenBank Accession No. NM_{—}000022.2) or VHL (von hippel lindau, NCBI GenBank Accession No. NM_{—}198156.1) gene can be additionally measured to diagnose autoimmune diseases. That is, autoimmune diseases can be diagnosed by measuring the methylation level of 1) CD3Z gene, 2) CD3Z and ADA genes, 3) CD3Z and VHL genes, or 4) CD3Z, ADA, and VHL genes. According to one embodiment of the present invention, hypermethylation of CD3Z, ADA, and VHL genes were found in rheumatoid arthritis, systemic lupus erythematosus or systemic sclerosis, compared to a normal control group (FIG. 4).

As used herein, the term “CD3Z (CD3 zeta, NCBI GenBank Accession No. NM_{—}190853.2)” is a gene known as CD247, and plays an important role in coupling antigen recognition to several intracellular signal-transduction pathways. Low expression of the antigen is known to result in impaired immune response. Protein expression of TCR, which is a product of the CD3Z gene, was reported to significantly decrease in peripheral T cells from patients with SLE. Mutations in the C-terminal SH2 (SRC homology) domain of the PTK ZAP70 protein, also known as TCRζ associated protein, were found in SKG mouse which is one of animal models of another autoimmune disease, rheumatoid arthritis (RA). Thus, it was also known that the TCRζ signal transduction is implicated in incidence or progression of RA. ZAP70 Mutations were not found in other RA animal models or RA patients, but down-regulation of TCRζ expression was found in T cells from patients with RA. The down-regulation of TCRζ expression was also found in T cells from patients with cancer as well as autoimmune diseases. It was suggested that the down-regulation of TCRζ expression induces T cell dysfunction to help tumors to escape from immune surveillance of the patient. This description is based on the fact that both of TCRζ down-regulation and T cell dysfunction are found in immune suppression induced by tumor cell-secreted factors. TCRζ down-regulation was also found in infectious diseases such as AIDS and leprosy, in which TCR devoid of ζ cannot be transported to the plasma membrane, and those are degraded in lysosomes, thereby reducing T cell function. It was reported that in cancer or SLE patients, FceRg associated with TCR, instead of ζ, can be transported to the plasma membrane, and a defect in T cell function was found.

As described above, TCRζ down-regulation is found in various diseases including autoimmune diseases, and there is also a close relationship between loss of TCRζ expression and T-cell function. Thus, many studies have been made on the mechanisms of TCRζ down-regulation for the treatment of autoimmune diseases. In a mouse tumor model, macrophage was found to decrease ζ chain expression, and also impair T cell function, suggesting that cytokines secreted by inflammatory cells including macrophage decrease TCRζ expression of T cells. It was reported that the cytokines, IFN-g and TNF play an important role in the reduction of ζ chain expression.

Another mechanism explains that degradation of arginine by arginase-1 secreted by MSC (myeloid suppressor cell) or macrophage decreases TCRζ expression of T cells. Arginine is known to play a central role in T cell proliferation and immune function, and macrophage and MSC migrate toward sites of inflammation such as tumor cell proliferation, graft versus host reaction, and infectious diseases. It was suggested that these cells secrete arginase-1 to degrade and reduce arginine, thereby decreasing TCRζ chain expression and T cell function.

Further, studies have been made to investigate TCRζ chain expression in T cells affected by cytokines, and reported that cytokines do not affect CD3Z mRNA level in T cells, but lysosomal degradation of TCRζ actively occurs to reduce the TCRζ chain, or TCRζ is a substrate for cleavage by protease such as caspase 3, and thus it is reduced by increasing caspase 3 activity in activated T cells. In addition to the protein reduction at the post-translational level, changes in TCRζ expression at the transcription level was found in SLE patients.

There have been many studies on the mechanisms of TCRζ down-regulation, but there is little report that changes in TCRζ expression could be caused by promoter CpG island hypermethylation of CD3Z gene. Moreover, the promoter CpG island hypermethylation of CD3Z gene has not been described in DNAs isolated from the whole blood of patients with autoimmune diseases.

According to one embodiment of the present invention, promoter hypermethylation of CD3Z gene was significantly increased in DNAs from the whole blood of SLE patients relative to healthy controls. Therefore, the present inventors newly suggested that promoter methylation of CD3Z gene can be used as a marker for prognosis or diagnosis of autoimmune diseases including SLE. Especially, each approximately 100 of SLE patients and control groups were made into one group, and then the promoter CpG island methylation level of CD3Z gene corresponding to the median value was regarded as a cutoff value for calculation of odds ratio (OR) for each disease. SLE showed an odds ratio of 29.78, and RA showed an odds ratio of 20.86 (Table 2), suggesting that changes in the CD3Z methylation can be used as a marker for prognosis or diagnosis of autoimmune diseases including SLE. Potential of the CD3Z methylation level as a marker for prognosis or diagnosis of autoimmune diseases was first demonstrated by the present inventors.

As used herein, the term “ADA (Adenosine deaminase, NCBI GenBank Accession No. NM_{—}000022.2)” exists in two main isoenzyme forms (ADA1, ADA2), and about 80% serum ADA are ADA2, originating from lymphocytes and monocytes. In acute hepatitis, ADA1 increases at the early stage, and ADA2 increases at the convalescent stage. Its activity also increases in chronic hepatitis, and increases more in cirrhosis. ADA1 increases in leukemia, whereas ADA2 increases in T-cell leukemia. ADA activity increases in infectious mononucleosis, rubella, and tuberculosis, and it originates from T-cell lymphocytes. ADA activity in newborn infants is the same as that in adults, and that in infancy is 1.5-2 times higher than that in adults, and gradually decreases. Hemolysis increases ADA, and it is not affected by anticoagulants. It was reported that ADA activity increases in the serum of SLE patients, and ADA2 of the two isoenzyme forms mainly increases. There has been no report on a relationship between promoter hypermethylation of ADA gene and autoimmune diseases, so far. The present inventors first demonstrated a relationship between ADA hypermethylation and autoimmune diseases including SLE.

As used herein, the term “VHL (Von Hippel Lindau, NCBI GenBank Accession No. NM_{—}198156.1)” is a gene encoding a tumor suppressor protein. Von Hippel Lindau disease is inherited in an autosomal dominant pattern, and occurs resulting from a germline mutation in the VHL gene. Germline mutation in the VHL gene leads to the development of benign and malignant tumors in the central nervous system and other organs, including the cerebellum, brainstem, spinal cord and retinal hemangioblastomas, renal cell carcinoma, pheochromocytoma and insulinoma. However, a relationship between VHL gene and autoimmune diseases has not been described yet. The present inventors first demonstrated a relationship between VHL hypermethylation and autoimmune diseases.

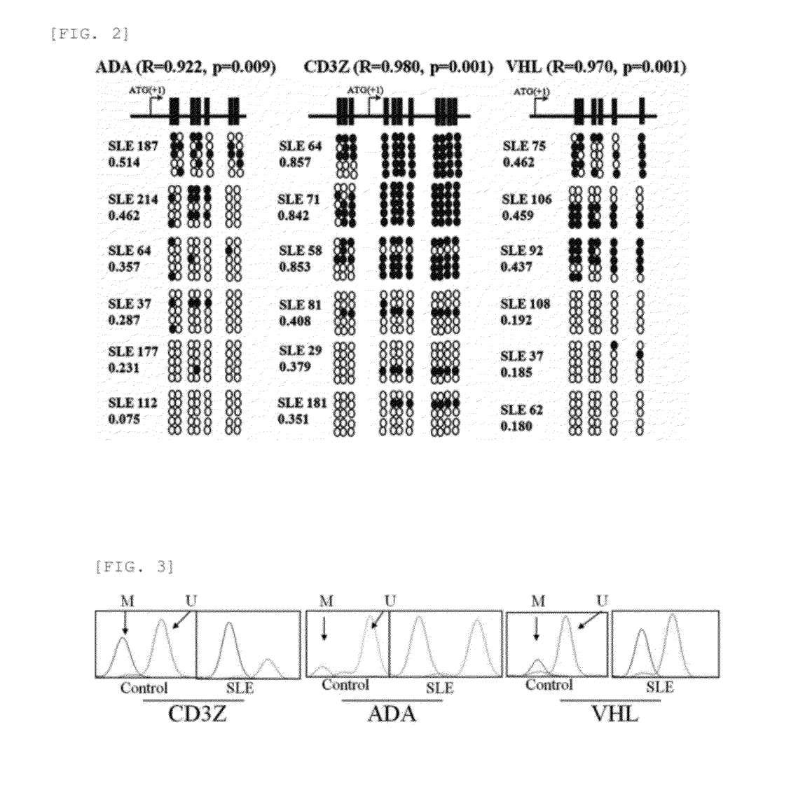

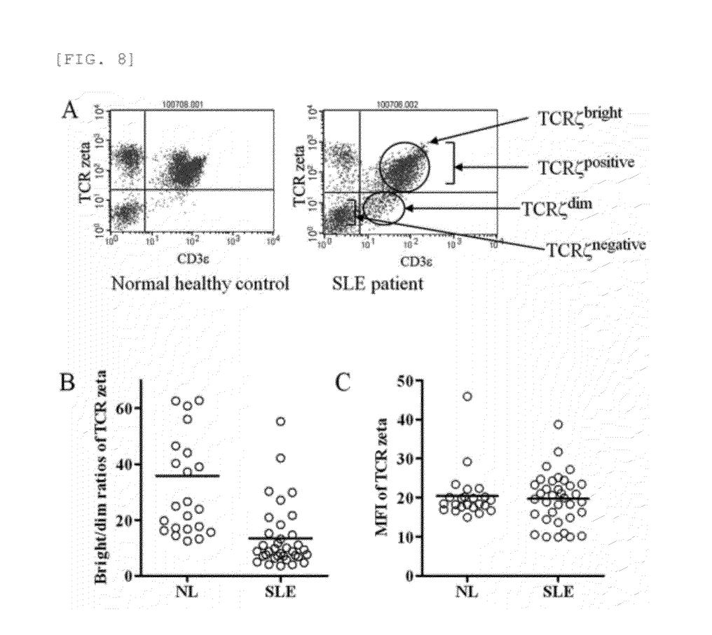

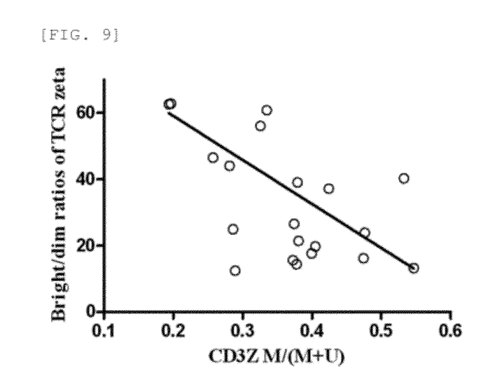

In a specific Example of the present invention, Illumina HumanMethylation27 BeadChip was used to analyze methylation. As a result, significant methylation levels of the CD3Z, ADA, and VHL genes were observed in autoimmune diseases. For quantitative analysis of the CD3Z, ADA, and VHL genes showing hypermethylation levels, the amplified products from bisulfite-modified DNAs were subjected to MSBE (methylation-specific single base extension). As a result, SLE patients showed higher promoter CpG island methylation than the control group (FIGS. 2 to 4). TCRζ down-regulation by methylation of CD3Z gene was observed (FIG. 8). When a demethylating agent, 5-AzadC was treated to the lung cancer cell lines, the reduced expression of CD3Z, ADA and VHL genes was restored (FIGS. 7A to C). These results suggest that the methylation of CD3Z, ADA and VHL genes can be used as a diagnostic marker for autoimmune diseases.

As used herein, the term “methylation” means a change in gene expression pattern by attachment of methyl groups to bases. With respect to the objects of the present invention, the methylation includes a methylation that occurs in the CD3Z gene, or any one or more of the CD3Z, ADA and VHL genes. Specifically, the methylation of the present invention includes a methylation that occurs in cytosines of CpG islands where C and G are at consecutive bases in the base sequences of any one or more of ADA, VHL and CD3Z genes, and therefore inhibits expression of a particular gene directly by blocking binding to DNA of transcription factors.

As used herein, the phrase “measuring a methylation level” means to determine the methylation level of a nucleic acid sequence, and with respect to the objects of the present invention, it means to determine the methylation level of CD3Z gene or any one or more of CD3Z, ADA and VHL genes. The measurement of a methylation level may be performed by any method of measuring a methylation level known in the art without limitation, and examples thereof include Illumina HumanMethylation27 BeadChip, Goldengate Methylation Cancer Panel I microarray, EpiTYPER™ analysis, MSBE (methylation-specific single base extension), or methylation-specific PCR (methylation-specific polymerase chain reaction), automatic sequencing or the like. The ADA, VHL or CD3Z methylation level was compared between blood samples of normal controls and those of autoimmune diseases patients, and then autoimmune diseases can be diagnosed depending on the methylation of ADA, VHL or CD3Z gene.

Preferably, the measurement of CD3Z methylation level may be performed by using a diagnostic composition for autoimmune diseases, including an agent measuring a promoter CpG island methylation level of the gene. More preferably, the composition may be a composition including an agent further measuring a promoter CpG island methylation level of ADA or VHL gene.

As used herein, the term “CpG island” refers to a genomic region that contains a high frequency of CpG, and it has a C+G content of more than 50% and a CpG ratio of more than 3.75%, and is 0.2-3 kb in length, wherein C represents cytosine, G represents guanine, and p means a phosphodiester bond between the cytosine and the guanine. There are about 45,000 CpG islands in the human genome, and they are mostly found in promoter regions regulating the expression of genes. Actually, the CpG islands occur in the promoters of housekeeping genes accounting for about 50% of human genes. In the somatic cells of normal persons, the CpG islands of such housekeeping gene promoter sites are un-methylated, but imprinted genes and the genes on inactivated X chromosomes are methylated such that they are not expressed during development. In the genomic DNA of mammal cells, there is the fifth base in addition to A, C, G and T, which is 5-methylcytosine where a methyl group is attached to the fifth carbon of the cytosine ring (5-mC). 5-mC is always attached only to the C of a CG dinucleotide (5′-mCG-3′), which is generally marked CpG. The C of CpG is mostly methylated by attachment with a methyl group. The methylation of this CpG inhibits a repetitive, sequence in genomes or transposon. Also, this CpG is a site where an epigenetic change in mammal cells occurs most often. The 5-mC of this CpG is naturally deaminated to thymine (T). With respect to the objects of the present invention, the promoter CpG hypermethylation of CD3Z gene can be determined as autoimmune diseases. The promoter CpG hypermethylation of ADA or VHL gene is additionally observed, autoimmune diseases can be more exactly determined.

The term “diagnosis”, as used herein, refers to evaluation of the presence or properties of pathological states. With respect to the objects of the present invention, the diagnosis is to determine the incidence of autoimmune diseases.

The autoimmune disease of the present invention to be diagnosed means a disease that is attributed to dysfunctional immune responses, leading to self-destruction. Examples thereof include, but are not limited to, systemic lupus erythematosus (SLE), rheumatoid arthritis (RA), sclerosis, systemic sclerosis

(SSC), polymyositis, dermatomyositis, anaphylactoid purpura, and Sjogren's syndrome. Preferably, the autoimmune disease may be rheumatoid arthritis, systemic lupus erythematosus or systemic sclerosis, and more preferably, systemic lupus erythematosus.

In the present invention, the agent measuring a methylation level of a gene may include a compound modifying an unmethylated cytosine base or a methylation-sensitive restriction enzyme, primers specific to the methylated sequence of the gene, and primers specific to the unmethylated sequence of the gene.

In the present invention, the agent measuring a methylation level of a gene may include a compound modifying an unmethylated cytosine base, a set of primers specific to the modified sequence of the gene, and extension primers.

The compound modifying an unmethylated cytosine base may be bisulfite, but is not limited thereto, preferably sodium bisulfite. A method of detecting promoter methylation by modifying the unmethylated cytosine residue using bisulfite is widely known in the art.

Further, the methylation-sensitive restriction enzyme is a restriction enzyme capable of specifically detecting CpG island methylation, and preferably a restriction enzyme including CG as a restriction enzyme recognition site. Examples thereof include SmaI, SacII, EagI, HpaII, MspI, BssHII, BstUI, NotI or the like, but are not limited thereto. Cleavage by a restriction enzyme differs depending on methylation or unmethylation of C at the restriction enzyme recognition site, and the methylation can be detected by PCR or Southern blot analysis. In addition to the restriction enzymes, other methylation-sensitive restriction enzymes are well known in the art.

As the method of quantitatively detecting CpG island methylation, pyrosequencing and methyl light methods are well known in the art. The pyrosequencing method is a method of determining a base sequence after PCR amplification of bisulfite-modified genomic DNA using primers specific thereto, and at this time, a relative amount of the methylated cytosine and thymine converted from the unmethylated cytosine can be exactly measured. Unlike Sanger sequencing, the pyrosequencing method is a method based on the detection of released pyrophosphate during DNA synthesis, and applied to quantification of methylation because it is more quantitative than the fluorescent dideoxynucleotide terminator method according to Sanger. In the methyl light method, primers are designed for amplification of only methylated cytosine during amplification of specific sequence of modified genomic DNA and labeled with fluorescence to detect PCR amplification. The method of quantitatively detecting CpG island methylation is not limited to these two methods. In addition, other method are also well known in the art: After PCR amplification of bisulfite-modified genomic DNA, the sequence converted to thymine is distinguished from the unmodified sequence due to methylation in the amplified sequence using restriction enzymes or mass measurement is performed by MALDI-TOF MS, thereby determining the relative amount.

Therefore, the agent of the present invention may include primers specific to methylated allele and unmethylated allele of ADA, VHL or CD3Z gene of patients suspected of having autoimmune diseases. Further, the agent of the present invention may include a set of primers specific to the allele of the modified gene of ADA, VHL or CD3Z gene of patients suspected of having autoimmune diseases, and extension primers.

The term “primer”, as used herein, means a short nucleic acid sequence having a free 3′ hydroxyl group, which is able to form base-pairing interaction with a complementary template and serves as a starting point for replication of the template strand. A primer is able to initiate DNA synthesis in the presence of a reagent for polymerization (i.e., DNA polymerase or reverse transcriptase) and four different nucleoside triphosphates at suitable buffers and temperature. In addition, the primers are sense and antisense nucleic acids having a sequence of 7 to 50 nucleotides. The primer may have additional properties that do not change the nature of the primer to serve as a starting point for DNA synthesis.

The primers of the present invention can be designed according to the CpG island sequence that is subjected to methylation analysis, and may be a set of primers that are able to specifically amplify bisulfite-unmodified cytosine due to methylation and a set of primers that are able to specifically amplify bisulfite-modified cytosine due to unmethylation, and also a set of primers specific to the modified sequence and extension primers. Preferably, with respect to the primers specific to the modified sequence of the gene, the primers for ADA may be a set of primers represented by SEQ ID NOs. 1 and 2, the primers for VHL may be a set of primers represented by SEQ ID NOs. 3 and 4, and the primers for CD3Z may be a set of primers represented by SEQ ID NOs. 5 and 6. Preferably, with respect to the extension primers of the gene, the primer for ADA may be a primer represented by SEQ ID NO. 7, the primer for VHL may be a primer represented by SEQ ID NO. 8, and the primer for CD3Z may be a primer represented by SEQ ID NO. 9.

The composition for diagnosis or prognosis of autoimmune diseases may further include polymerase, agarose, and a buffer solution for electrophoresis, in addition to the above agent.

In still another embodiment, the present invention relates to a method for diagnosing autoimmune diseases, comprising the steps of measuring a methylation level of CD3Z gene (CD3 zeta, NCBI GenBank Accession No. NM_{—}198053.2) in a biological sample of a patient suspected of having autoimmune diseases; and comparing the methylation level to that of the corresponding gene in a normal control group. Preferably, the methylation measurement may further include a step of measuring and comparing a methylation level of ADA or VHL gene.

Preferably, the autoimmune disease is rheumatoid arthritis, systemic lupus erythematosus, or systemic sclerosis, and more preferably systemic lupus erythematosus.

The step of measuring the methylation levels of any one or more of ADA, VHL, and CD3Z genes includes a step of measuring promoter CpG island methylation levels of the genes.

The term “biological sample”, as used herein, includes samples displaying a difference in the methylation levels of one or more of ADA, VHL and CD3Z genes by the incidence of autoimmune diseases, such as tissues, cells, whole blood, serum, plasma, saliva, sputum, or urine, but is not limited thereto. Preferably, the biological sample of the present invention may be a genomic DNA of the blood cells of a patient.

First, in the step of measuring the methylation level of DNAs obtained from the patients suspected of having autoimmune diseases, the genomic DNAs can be obtained by a phenol/chloroform extraction method, an SDS extraction method (Tai et al., Plant Mol. Biol. Reporter, 8: 297-303, 1990), a CTAB separation method (Cetyl Trimethyl Ammonium Bromide; Murray et al., Nuc. Res., 4321-4325, 1980) typically used in the art, or using a commercially available DNA extraction kit.

The step of measuring the methylation level of the gene may include the steps of a) treating the obtained genomic DNA with a compound modifying unmethylated cytosine or a methylation-sensitive restriction enzyme; and b) amplifying the treated DNA by PCR using primers capable of amplifying the gene.

In step a), the compound modifying unmethylated cytosine may be bisulfite, and preferably sodium bisulfite. The method of detecting promoter methylation by modifying unmethylated cytosine residues using bisulfite is widely known in the art. Further, in step a), the methylation-sensitive restriction enzyme is, as described above, a restriction enzyme capable of specifically detecting CpG island methylation, and preferably a restriction enzyme containing CG as a restriction enzyme recognition site. Examples thereof include SmaI, SacII, EagI, HpaII, MspI, BssHII, BstUI, NotI or the like, but are not limited thereto.

In step b), the amplification may be performed by a typical PCR method. The primers used herein are, as described above, designed according to the CpG island sequence that is subjected to methylation analysis, and may be a set of primers that are able to specifically amplify bisulfite-unmodified cytosine due to methylation and a set of primers that are able to specifically amplify bisulfite-modified cytosine due to unmethylation, and also a set of primers specific to the modified sequence of the gene and extension primers.

The step of measuring the methylation level of the ADA, VHL or CD3Z gene may further include the step of c) identifying the presence of a product amplified in step b).

In step c), the presence of the amplified product may be identified by a method known in the art. For example, electrophoresis is performed to detect the presence of a band at the desired size. For example, in the case of using the compound modifying the unmethylated cytosine residues, methylation can be determined according to the presence of the PCR product that is amplified by the two types of primer pairs used in step a), that is, the set of primers that are able to specifically amplify bisulfite-unmodified cytosine due to methylation and a set of primers that are able to specifically amplify bisulfite-modified cytosine due to unmethylation. Further, methylation can be quantitatively determined by the set of primers specific to the modified sequence and extension primers.

Preferably, methylation can be quantitatively determined by treating genomic DNA of a sample with sodium bisulfite, specifically amplifying bisulfite-modified cytosine of ADA, VHL or CD3Z gene, and then analyzing the amplified base sequence by single base extension.

Further, in the case in which a restriction enzyme is used, methylation can be determined by a method known in the art. For example, when the PCR product is present in the restriction enzyme-treated DNA, under the state where the PCR product is present in the mock DNA, it is determined as promoter methylation. When the PCR product is absent in the restriction enzyme-treated DNA, it is determined as promoter unmethylation. Accordingly, the methylation can be determined, which is apparent to those skilled in the art. The term ‘mock DNA’ refers to a DNA isolated from clinical samples with no treatment. Therefore, the method of providing information for the diagnosis of autoimmune diseases of the present invention is used to effectively examine the methylation of ADA, VHL or CD3Z gene, thereby diagnosing autoimmune diseases.

In still another embodiment, the present invention relates to a diagnostic kit for autoimmune diseases, comprising the above composition. Preferably, the diagnostic kit for autoimmune diseases may be composed of a composition, solution or apparatus, which includes one or more kinds of different constituents suitable for analysis methods. The autoimmune diseases are preferably rheumatoid arthritis, systemic lupus erythematosus, or systemic sclerosis, and more preferably systemic lupus erythematosus.

Hereinafter, the present invention will be described in more detail with reference to Examples. However, these Examples are for illustrative purposes only, and the invention is not intended to be limited by thereby.

### EXAMPLE 1

### Preparation of Blood DNA Samples of SLE (Systemic Lupus Erythematosus) Patients, Rheumatoid Arthritis Patients, Systemic Sclerosis Patients, Healthy Controls, and Twin Controls

The present invention includes blood DNA samples isolated from 105 healthy non-autoimmune disease control, 108 active SLE patients, 6 non-active SLE patients, 19 rheumatoid arthritis patients, 18 systemic sclerosis patients, and 20 breast cancer patients, and also blood DNA samples isolated from 4 pairs of twins discordant for SLE. All patients with autoimmune diseases of SLE, rheumatoid arthritis, and systemic sclerosis satisfied the American College of Rheumatology classification criteria for each disease. Non-active SLE patients had a disease activity index (DAI) of 2 or less, and most of the active SLE patients participating in the study had an SLE DAI of 5 or more. The study protocol was approved by the Institutional Review Board and the Ethics Committee of Seoul National Hospital. Peripheral venous blood was collected from SLE attending the Seoul National University Hospital (aged between 18 and 56 years, mean), healthy control patients (aged between 23 and 45 years, mean), and twin pairs.

### EXAMPLE 2

### Methylation Assay Using Illumina HumanMethylation27 BeadChip

Genomic DNAs from six SLE patients and six control patients were used for the analysis. Illumina HumanMethylation27 BeadChip was used to analyze methylation. As a result, among the genes showing significant changes in the level of CpG island methylation between SLE patients and controls, three genes including CD3Z, ADA, and VHL, whose methylation level increased in SLE patients, were chosen for the validation of the methylation array results.

### EXAMPLE 3

### Preparation of Blood DNA Sample from Whole Blood

Genomic DNA was isolated from heparinized whole blood for quantitative analysis of promoter methylation. For isolation of genomic DNA, a DNA purification kit provided by Qiagen was used.

### EXAMPLE 4

### Quantitative Analysis of Promoter CpG Island Methylation

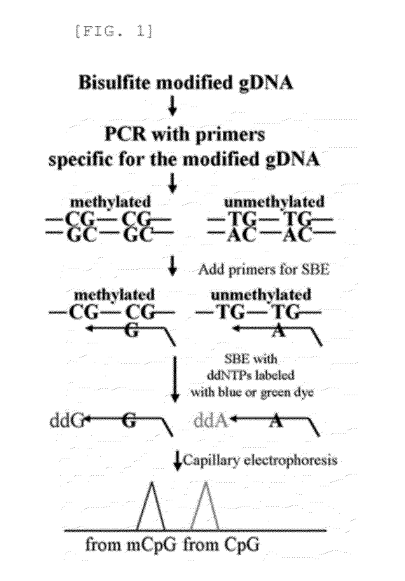

Among genes showing significant difference of methylation levels between SLE patients and controls, three genes obtained in Example 2, whose methylation level increased in the blood of SLE patients relative to control patients, were chosen for the validation of the methylation array results. The CpG island regions for three genes of CD3Z, ADA and VHL were determined based on the result of Illumina HumanMethylation27 BeadChip array. First, the methylation status of CD3Z, ADA, and VHL was determined by single base extension method after amplification of a particular gene by bisulfite-modified genomic sequencing, as in a previous method (Hong K M, et al., Semiautomatic detection of DNA methylation at CpG islands. Biotechniques. 38(3):354, 356, 358. (2005)). The principle of the single base extension method is shown in FIG. 1. About 1 μg of genomic DNA was used in the sodium bisulfite treatment, as described in the Zymo Research's instructions. A DNA sample was mixed with M-dilution buffer for denaturation, and was incubated at 37° C. for 15 minutes. CT Conversion Reagent was added to the denatured DNA and incubated in darkness at 50° C. for 12-16 hours to convert unmethylated cytosine to thymidine in the DNA sequence. After the modification, an M-binding buffer was added, and the mixture was loaded onto a Zymo-Spin I column. After washing with an M-Wash buffer, the modified DNA was desulfonated to complete the modification reaction. After further washing, the modified DNA was eluted with 20 μL M-Elution buffer. The modified genomic DNA was used to amplify specific DNA products with gene-specific primers for the sodium bisulfite-treated DNA sequences (Table 1).

TABLE 1

| | |
|---|---|
| ADAmF | GTTGGYGTATAAAGTTGTTTTTATTTATTGAGTATTTAGTAG, |
| | Y = C/T |
| | (SEQ ID NO. 1) |
| ADAmR | CTCAATTTCRCTATCTATCAAATAAACATCCTAACAC, |
| | R = G or A |
| | (SEQ ID NO. 2) |
| ADA- | CTACICCIAATAAACACCTAATACTATACCCIAC, |
| SBE1 | I = inosine |
| | (SEQ ID NO. 7) |
| VHLmF | GATAGATGTAAAGATAGGAATAAGTTAGGGTTATG |
| | (SEQ ID NO. 3) |
| VHLmR | CTCCRAAAAATACTCCTTAACTAAAACCACAC, |
| | R = G or A |
| | (SEQ ID NO. 4) |
| VHL-E1 | GATGTAAAGATAGGAATAAGTTAGGGTTATGTTGG |
| | (SEQ ID NO. 8) |
| CD3ZmF | TTTGGGGAGGTAGTTGTAGAATAAAATTAGTAG |
| | (SEQ ID NO. 5) |
| CD3ZmR | CACACCCACTCCCCTACACATACAC |
| | (SEQ ID NO. 6) |
| CD3Z-E1 | GGGAAAGGATAAGATGAAGTGGAAGG |
| | (SEQ ID NO. 9) |

PCR amplification was performed by means of initial incubation at 95° C. for 10 minutes, followed by 35 cycles of 95° C. for 30 seconds, 56° C. for 30 seconds, and 72° C. for 1 minute, with a final extension at 72° C. for 30 minutes in a mixture containing 1×PCR buffer II™ buffer (Roche) with 1.5 mM MgCl₂, 0.2 mM dNTPs, pmol of each primer, and 50-100 ng of bisulfite-treated genomic DNA. The amplified products (20 μL) were then purified using a QIAquick™ PCR Purification Kit (Qiagen) and eluted in a final volume of 30 μL. After purification, a single base extension was performed using a SNaPshot kit (Applied Biosystems) and the single base extension primers for each gene (Table 1).

The single base extension was performed under the following conditions: 20 cycles of 96° C. for 10 seconds, 50° C. for 5 seconds, and 60° C. for 30 seconds, and a product of the single base extension was analyzed using an ABI 3100 sequencer (Applied Biosystems).

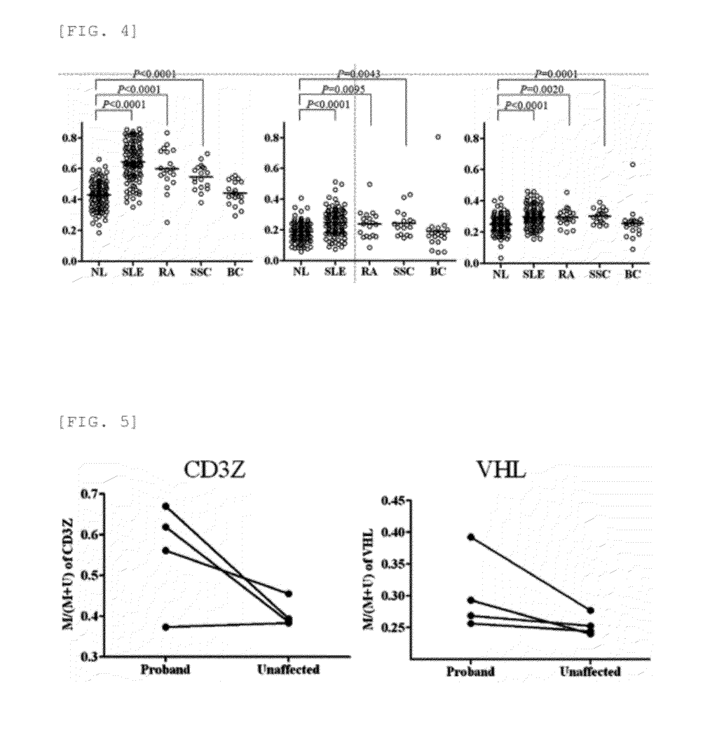

As a result, three of the CD3Z, ADA and VHL genes showed significant changes in the DNA methylation level, compared to a control group, in a screening study of DNAs obtained from SLE patients (FIG. 2). In the base sequence determination, C was determined as methylated, and T was determined as unmethylated. Methylated CpGs are indicated by the filled circles, and unmethylated CpGs are indicated by the open circles. For quantitative comparison between MSBE and bisulfite sequencing, the methylation levels {M/(M+U)} obtained from the MSBE results of six samples were marked in the side of each sample number, and the results are compared to the percentages of the filled circles (M and U represent signal intensities of methylated and unmethylated peaks, respectively and M/(M+U) represents a methylation level). A significant correlation was observed in all three genes of CD3Z, ADA, and VHL (Pearson correlation coefficient R=0.922(ADA), 0.980(CD3Z) or, 0.970(VHL) p=0.009(ADA), 0.001(CD3Z), or 0.001(VHL)). For further confirmation of hypermethylation for three genes, CD3Z, ADA and VHL, the amplified products of CpG island sequences from bisulfite-modified gDNAs of SLE patients and controls were cloned and sequences were determined. As shown in FIG. 2, SLE patients had higher promoter CpG island methylation than those from control, which is in agreement with the result from MSBE method. The MSBE results of CpG island methylation of CD3Z, ADA and VHL are represented by signal intensities of methylated (M) and unmethylated (U) peaks and the results of quantitative analysis are shown in FIG. 3. Increased promoter methylation was observed in SLE patients (SLE1, SLE2 and SLE3).

In addition, an odds ratio (OR) for SLE was calculated using the methylation levels of CD3Z, ADA, and VHL, namely, M/(M+U) values, and shown in the following Table 2. The patient groups and the control groups were made into one group, and a median value of the methylation levels of each gene was obtained, and a value below the median value was determined as negative, and a value above the median value was determined as positive for calculation of OR. With respect to SLE, CD3Z showed an odds ratio of 29.78, and ADA showed an odds ratio of 3.39, VHL showed an odds ratio of 4.75. With respect to SSC, CD3Z showed an odds ratio of 8.74, and ADA showed an odds ratio of 4.78, VHL showed an odds ratio of 4.36. With respect to RA, CD3Z showed an odds ratio of 20.86, and ADA showed an odds ratio of 3.15, VHL showed an odds ratio of 4.73.

TABLE 2

| | CD3Z+ | CD3Z− | OR for CD3Z |
|---|---|---|---|
| SLE | 91 | 17 | 29.78 |
| Control | 16 | 89 | |
| | ADA+ | ADA− | OR for ADA |
| SLE | 70 | 38 | 3.39 |
| Control | 37 | 68 | |
| | VHL+ | VHL− | OR for VHL |
| SLE | 74 | 34 | 4.75 |
| Control | 33 | 72 | |
| | CD3Z+ | CD3Z− | OR for CD3Z |
| SSC | 11 | 7 | 8.74 |
| Control | 16 | 89 | |
| | ADA+ | ADA− | OR for ADA |
| SSC | 13 | 5 | 4.78 |
| Control | 37 | 68 | |
| | VHL+ | VHL− | OR for VHL |
| SSC | 12 | 6 | 4.36 |
| Control | 33 | 72 | |
| | CD3Z+ | CD3Z− | OR for CD3Z |
| RA | 15 | 4 | 20.86 |
| Control | 16 | 89 | |
| | ADA+ | ADA− | OR for ADA |
| RA | 12 | 7 | 3.15 |
| Control | 37 | 68 | |
| | VHL+ | VHL− | OR for VHL |
| RA | 13 | 6 | 4.73 |
| Control | 33 | 72 | |

Moreover, quantitative changes in the promoter CpG island methylation levels of CD3Z (FIG. 4A), ADA (FIG. 4B) and VHL (FIG. 4C) were compared. SLE patients showed significantly high values (p<0.01 by Wilcoxon rank sum test) in the promoter CpG island methylation of three different genes, compared to the healthy control group (NL). The Y axis represents a signal ratio of a methylated peak (M) to an unmethylated peak (U). In addition to SLE, rheumatoid arthritis (RA) and systemic sclerosis (SSC) patients also showed significantly high values (p<0.01 by Wilcoxon rank sum test) in the promoter CpG island methylation of three different genes, compared to the healthy control group. These results indicate that autoimmune diseases can be diagnosed by measuring methylation levels of the genes.

The promoter methylation levels of CD3Z and VHL were also examined in 4 pairs of twins discordant for SLE. Although there is no statistical significance because of the small numbers involved, increased methylation levels were observed in SLE patients, compared to non-patient siblings (FIG. 5). The SLE incidence may differ in genetically identical monozygotic twins, suggesting that SLE can be also associated with epigenetic changes other than inheritance.

### EXAMPLE 5

### Human Blood Cell Subfractionation and Promoter Methylation Analysis

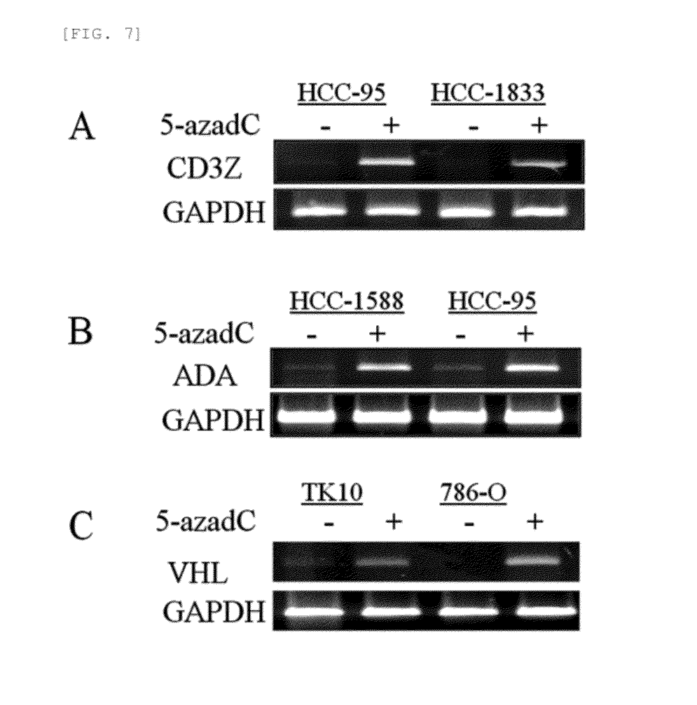

Peripheral blood cells were obtained and RBC was underwent lysis using an RBC lysis buffer from heparinized peripheral venous blood immediately following venesection. Specifically, the RBC lysis buffer (Roche) was first used to lyse RBC from the whole blood, and then mononucleated and polynucleated cell fractions were separated by Ficoll-Hypaque density gradient centrifugation (Sigma). The separated middle layer was used as a mononucleated cell fraction including lymphocytes, and the bottom layer was used as a polynucleated cell fraction including RBC. The promoter methylation of CD3Z, ADA, and VHL genes was examined, when SLE blood cells were separated into the whole blood (WB), the mononucleated cell (MNC) and the granulocyte (Gran). As a result, there was a significant correlation between the methylation levels of CD3Z (FIG. 6A), ADA (FIG. 6B), and VHL (FIG. 6C) in the whole blood and those in mononucleated or polynucleated cells. The level of significance between the methylation levels of ADA and VHL in the whole blood and those in mononucleated or polynucleated cells was p<0.0001 at Spearman test. The level of significance between the methylation levels of CD3Z in the whole blood and those in mononucleated cells was p=0.001, and the significant correlation between the methylation levels of CD3Z in the whole blood and those in polynucleated cells was p=0.003 (FIG. 6).

Further, there were significant differences in the CD3Z methylation levels of the whole blood, mononucleated cells, and polynucleated cells between active SLE patients and non-active SLE patients (FIG. 6A; whole blood: p=0.0075, mononucleated cells: p=0.0047, polynucleated cells: p=0.0002). A significant increase in the VHL methylation was also observed in the whole blood (p=0.0225) and mononucleated cells (p=0.0002) of SLE patients (FIG. 6C). However, higher ADA methylation was observed in non-active SLE patients, and there was a statistically significant difference in mononucleated cells (FIG. 6B; p=0.042 by Mann Whitney test). These results indicate that disease activity of autoimmune diseases including SLE can be also examined by measuring the methylation levels of CD3Z, VHL and ADA genes.

### EXAMPLE 6

### Inverse Relationship Between CD3Z, ADA, and VHL Expression and CD3Z, ADA, and VHL Promoter Methylation

To test whether CD3Z, ADA, and VHL expressions are related to promoter CpG island methylation, a demethylating agent, 5-azadC (5-aza deoxy cytidine) was treated to two of the lung cancer cell lines.

Specifically, promoter CpG island methylation of CD3Z gene in HCC-95 and HCC-1833 cell lines, that of ADA gene in HCC-95 and HCC-1588 cell lines, and that of VHL gene in TK10 and 786-O cell lines were observed, and in each case, there was no expression of each gene. However, the expression of each gene was restored after 48-hour treatment of the cell lines with a demethylating agent 5-azadC. In this regard, GAPDH was used as a control (FIGS. 7A to 7C).

This result suggests that promoter CpG island methylation of the genes shows inhibitory effects on gene expression.

### EXAMPLE 7

### Human Blood Cell Subfractionation, FACS Analysis, and TCRζ Expression Analysis

7-1: Human Blood Cell Subfractionation and FACS Analysis

Peripheral blood cells were obtained and RBC underwent lysis using RBC lysis buffer from heparinized peripheral venous blood immediately following venesection. Specifically, the RBC lysis buffer (Roche) was first used to lyse RBC from the whole blood, and then PBMC and polymorphonuclear fractions were separated by Ficoll-Hypaque density gradient centrifugation (Sigma). The separated middle layer was used as a mononucleated cell fraction including lymphocytes, and the bottom layer was used as a polynucleated cell fraction including RBC. For FACS analysis, anti-CD3-PerCP and anti-TCRζ-PE were purchased from Immunotech (Beckman Coulter).

7-2: TCRζ Expression Analysis

Because the TCR-chain has a short, nine-amino acid extracellular domain, mAbs that detect the intracellular cytoplasmic domain epitopes after fixing and permeabilization were used to analyze TCRζ expression (TIA-2; Beckman Coulter). Surface staining of T cell subsets was performed by standard methods. Isotype-matched control Abs were used to confirm expression specificity. Analysis was performed with a FACSCalbur flowcytometer (BD Biosciences) and CellQuest software (BD Biosciences). The analysis is based on two independent sets of variables: 1) constitutive expression of TCRζ is determined by calculating the ratio of the mean fluorescence intensity (MFI) of the TCRζ^{positive} population to the fluorescence intensity (MFI) of the TCRζ^{negative} population, comprising B cells and monocytes; and 2) the ratio of the number of circulating ^{TCRζ}^{bright} to TCRζ^{dim} cells is calculated. The relationship between TCRζ expression and the degree of CD3Z methylation were analyzed using SPSS software. The MFI index is calculated by dividing the number of MFI TCRζ^{positive} cells by the number of MFI TCRζ^{negative} cells in A. The TCRζ^{bright}/TCRζ^{dim} ratio is calculated by dividing the number of TCRζ^{bright} cells by the number of TCRζ^{dim} cells.

The TCRζ expression index in SLE patients was determined by flow cytometry. Based on the MFI index and the TCRζ^{bright}/^{dim} ratio, the quantitative changes in the CD3 positive T cells with TCRζ expression was examined. As a result, down-regulation of CD3Z protein product in SLE was observed (FIG. 8A). For examination of TCRζ-chain, the expression levels of TCRζ-chain were examined in healthy controls and SLE patients. Compared to the normal healthy controls, there was no difference in the MFI index between SLE patients and controls (FIG. 8B), but a significant decrease in the TCRζ^{bright}/^{dim} ratio was observed in SLE patients (p<0.001, by Mann-Whitney U test) (FIG. 8C).

Further, the ^{TCRζ}^{bright}/^{dim} ratio of 21 healthy controls determined by flow cytometry was in inverse proportion to the CD3Z methylation level, namely, M/(M+U) (p=0.0140 by Spearman correlation test) (FIG. 9), indicating that the promoter methylation of CD3Z gene is inversely related to the TCRζ-chain expression, and the CD3 positive T cells with TCRζ expression is lower in patients with high level of CD3Z promoter methylation relative to patients with low level of methylation.

Taken together, the above results suggest that CD3Z methylation, or ADA, VHL methylation is specific to autoimmune diseases, and prognosis or diagnosis of autoimmune diseases can be achieved by measuring the methylation level of the genes.

### EFFECT OF THE INVENTION

As described above, hypermethylation of CD3Z gene or any one of CD3Z, ADA and VHL genes of the present invention is specific to autoimmune diseases, and thus a composition including an agent measuring the methylation level of the present invention can be used for prognosis or diagnosis of autoimmune diseases.

What is claimed is:

1. A diagnostic composition for autoimmune diseases, comprising an agent measuring a methylation level of CD3Z gene (CD3 zeta, NCBI GenBank Accession No. NM_{—}198053,2).

2. The diagnostic composition for autoimmune diseases according to claim 1, further comprising an agent measuring a methylation level of ADA (Adenosine deaminase, NCBI GenBank Accession No. NM_{—}000022.2) or VHL (von hippel lindau, NCBI GenBank Accession No. NM_{—}198156.1) gene.

3. The diagnostic composition for autoimmune diseases according to claim 1, wherein the agent measuring a methylation level of a gene includes a compound modifying an unmethylated cytosine base or a methylation-sensitive restriction enzyme, primers specific to the methylated sequence of the gene, and primers specific to the unmethylated sequence of the gene.

4. The diagnostic composition for autoimmune diseases according to claim 2, wherein the agent measuring a methylation level of a gene includes a compound modifying an unmethylated cytosine base or a methylation sensitive restriction enzyme, primers specific to the methylated sequence of the gene, and primers specific to the unmethylated sequence of the gene.

5. The diagnostic composition for autoimmune diseases according to claim 1, wherein the agent measuring a methylation level of a gene includes a compound modifying an unmethylated cytosine base, a set of primers specific to the modified sequence of the gene, and extension primers.

6. The diagnostic composition for autoimmune diseases according to claim 2, wherein the agent measuring a methylation level of a gene includes a compound modifying an unmethylated cytosine base, a set of primers specific to the modified sequence of the gene, and extension primers.

7. The diagnostic composition for autoimmune diseases according to claim 1, wherein the autoimmune disease is selected from the group consisting of rheumatoid arthritis, systemic lupus erythematosus, and systemic sclerosis.

8. The diagnostic composition for autoimmune diseases according to claim wherein the primers specific to the modified sequence of CD3Z gene are a set of primers represented by SEQ ID NOs. 5 and 6.

9. The diagnostic composition for autoimmune diseases according to claim 6, wherein with resect to the primers specific, to the modified sequence of the gene, the primers for ADA are a set of primers represented by SEQ ID NOs. 1 and 2, the primers for VHL are a set of primers represented by SEQ ID NOs. 3 and 4, and the primers for are a set of primers represented by SEQ ID NOs. 5 and 6.

10. The diagnostic composition for autoimmune diseases according to claim 5, wherein the extension primer of CD3Z gene is a primer represented by SEQ ID NO. 9.

11. The diagnostic composition for autoimmune diseases according to claim 6, wherein with respect to the extension primers of the gene, the primer for ADA is a primer represented by SEQ ID NO. 7, the primer for VHL is a primer represented by SEQ ID NO. 8, and the primer for CD3Z is a primer represented by SEQ ID NO. 9.

12. The diagnostic composition for autoimmune diseases according to claim 3, wherein the compound modifying an unmethylated cytosine base is sodium bisulfite.

13. The diagnostic composition for autoimmune diseases according to claim 5, wherein the compound modifying an unmethylated cytosine base is sodium bisulfite.

14. A method for diagnosing autoimmune diseases, comprising the steps of measuring a methylation level of CD3Z gene (CD3 zeta, NCBI GenBank Accession No. NM_{—}198053.2) in a biological sample of a patient suspected of having autoimmune diseases; and comparing the methylation level to that the corresponding gene in a normal control group.

15. The method according to claim 14, further comprising the step of measuring and comparing a methylation level of ADA (Adenosine deaminase, NCBI GenBank Accession. No NM_{—}000022.2) or VHL (von hippel lindau, NCBI GenBank Accession No. NM_{—}198156.1) gene.

16. The method according to claim 14, wherein the step of measuring the methylation level of the gene includes the steps of:
a) treating the obtained genomic DNA with a compound modifying unmethylated cytosine or a methylation-sensitive restriction enzyme; and
b) amplifying the treated DNA by PCR using primers capable of amplifying the gene.

17. The method according to claim 16, wherein the compound modifying unmethylated cytosine is sodium bisulfite, and the method of measuring the methylation level is methylation-specific single base extension (MSBE).

18. The method according to claim 14, wherein the autoimmune disease is selected from the group consisting of rheumatoid arthritis, systemic lupus erythematosus, and systemic sclerosis.

19. A diagnostic kit for autoimmune diseases, comprising the composition of claim 1.

20. A diagnostic kit for autoimmune diseases, comprising the composition of claim 2.
